(19) United States
(12) Patent Application Publication
(10) Pub. No.: US 2018/0333086 A1
(43) Pub. Date: Nov. 22, 2018

(54) CALCIUM SENSOR AND IMPLANT

(71) Applicant: Elenza, Inc., Roanoke, VA (US)

(72) Inventor: Amitava Gupta, Roanoke, VA (US)

(73) Assignee: Elenza, Inc., Roanoke, VA (US)

(21) Appl. No.: 15/775,238

(22) PCT Filed: Nov. 11, 2016
(86) PCT No.: PCT/US16/61715

§ 371 Date: May 10, 2018

Related U.S. Application Data
(60) Provisional application No. 62/253,945, filed on Nov. 11, 2015.

(51) Int. Cl. A61B 5/145; A61B 5/1486; A61B 5/1473; A61B 5/07; A61B 5/00; A61B 5/1459; G01N 27/414

(52) U.S. Cl. CPC A61B 5/14546; A61B 5/14865; A61B 5/14735; A61B 5/076; A61B 5/7282; A61B 2560/0219; G01N 27/414; A61B 2562/0285; A61B 2560/0481; A61B 2562/028; A61B 5/1459

(57) ABSTRACT

An implant includes a first calcium sensor portion and a body portion. The first calcium sensor portion includes a calcium-selective binding portion. The body portion includes a controller configured to process a signal received from the calcium-selective binding portion, and a transmitter/receiver.

### CROSS-REFERENCE TO RELATED PATENT APPLICATIONS

This application claims priority to U.S. Provisional Application 62/253,945, filed Nov. 11, 2015, entitled “DEVELOPMENT OF A UNIVERSAL SENSOR OF MUSCULAR RESPONSE ASSOCIATED WITH AN IMPLANT,” the entirety of which is incorporated herein by reference.

### BACKGROUND

It is known that a wide variety of muscular and neuronic responses are actuated by calcium ion expression. Some calcium ion sensors exist that can measure calcium ion concentrations in vitro. However, many calcium ion sensors are too large to use in conjunction with an implant in a person, or they may have a short shelf-life and would require surgical replacement after a short period of time.

### SUMMARY

In some embodiments, according to one aspect, an implant includes a first calcium sensor portion and a body portion. The first calcium sensor portion includes a calcium-selective binding portion. The body portion includes a controller configured to process a signal received from the calcium-selective binding portion, and a transmitter/receiver.

In some embodiments, according to another aspect a calcium ion sensor includes a calcium-selective binding portion, a power source, a transmitter/receiver, a controller, and a memory device.

### BRIEF DESCRIPTION OF THE DRAWINGS

FIG. 1A shows intracellular events.

FIG. 1B is a schematic representation of a Ca2+ cycle.

FIG. 1C shows calcium release in a muscle cell.

FIG. 2A shows results of calcium ion sensor measurements made using self-referencing ion selective probes.

FIG. 2B shows results of calcium ion sensor measurements made using self-referencing ion selective probes.

FIG. 3 depicts some embodiments of a calcium sensor implant.

FIG. 4 depicts some embodiments of a calcium sensor portion of an implant.

FIG. 5 is a block diagram depicting some embodiments of a calcium sensor implant.

FIG. 6 depicts a knee implant.

FIG. 7 depicts some embodiments of a calcium sensor implant that includes two calcium sensor portions.

FIG. 8 shows a cross section of silicon nanowire based field effect transistor sensors.

FIG. 9 shows an extended gate field effect transistor.

FIG. 10 shows cantilever sensor measurements.

### DETAILED DESCRIPTION

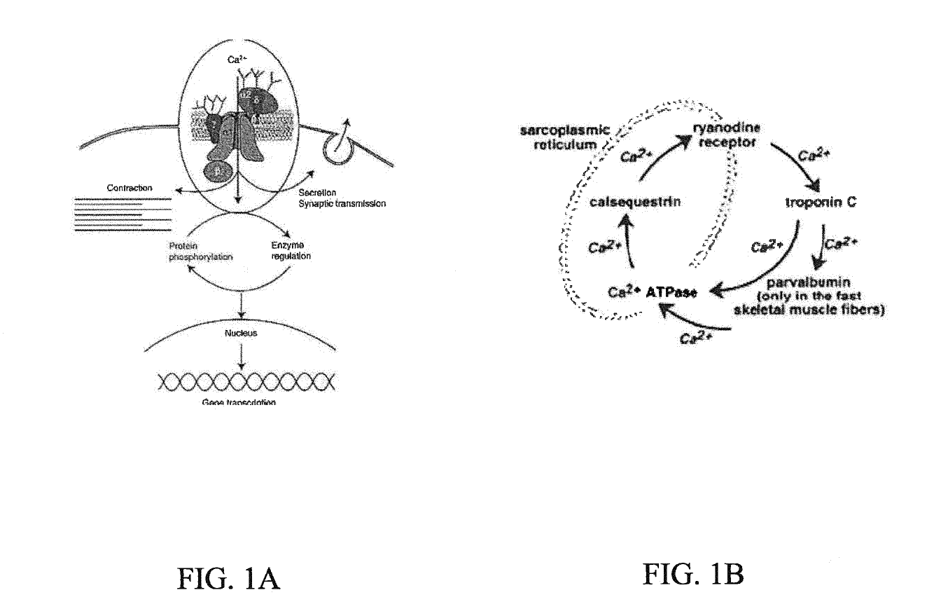

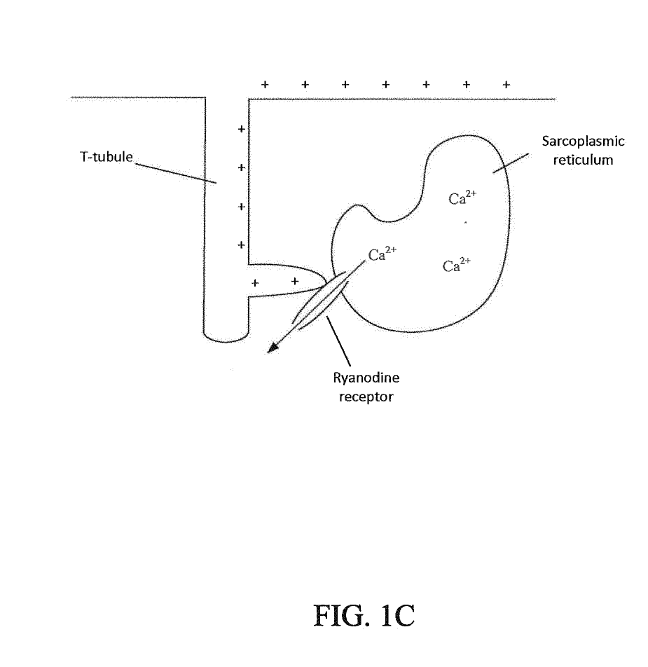

It is known that a wide variety of muscular and neuronic responses are actuated by calcium ion (Ca²⁺ ion) expression. FIG. 1A shows a wide range of intracellular events including contraction, secretion, enzyme regulation, synaptic transmission, and protein phosphorylation, all activated by Ca²⁺ ions entering a cell. (Catterall, W A, “Voltage gated calcium channels” in Cold Spring Harb Perspect Biol 2011; 3: a003947, Martin Bootman, Michael Berridge, James W Putney and Llewelyn Roderick eds.). FIG. 1B is a schematic representation of a Ca²⁺ cycle in muscle contraction and relaxation in skeletal muscles. Ca2+ cycle in contraction and relaxation in skeletal muscles. (Berchtold, M W, Brinkmeier, H, and Muntener, M, “Calcium Ion in Skeletal Muscle: Its Crucial Role for Muscle Function, Plasticity and Disease”, in Physiological Review, Vol 80, No 3, 2000; pp 1216-1251). Herein is disclosed a Ca²⁺ ion sensor that may be packaged with a variety of implants such as orthopedic implants, cardiac implants (e.g., stents), intraocular implants such as intraocular lenses and glaucoma shunts, and neural implants such as neurostimulators or brain shunts. Including a Ca²⁺ sensor with an implant allows for monitoring the functioning of the implant, integration of the implant with human tissue, muscular function adjacent to the implant or any chronic inflammatory responses that may be caused by the implant. In one embodiment, a Ca²⁺ sensor will include a microelectrode modified by a Ca²⁺ ion selective coating and incorporated into a field effect transistor. Other embodiments include calcium sensors that detect binding of free and complexed Ca²⁺ ions on the electrode surface in other manners.

Calcium Ion Channels and Calcium Surges

Calcium voltage-gated ion channels can be of L (HVA), T (LVA), or N type, and are found in skeletal muscle and heart muscle. There is also a fourth type of called “P type,” found in Purkinje cells. First isolated in 1984 from transverse tubules of skeletal muscle, P type calcium ion channels function primarily as voltage sensors for excitation-contraction coupling in muscle. Voltage-gated calcium channels are found in protozoans such as paramecium, as well as in almost every excitable cell in animals. They play a unique role in that they are involved in taking electrical signals and making chemical signals out of them. Some are involved in excitation while others are involved in regulation of secretion, contraction (e.g. of muscle) and gating (other ion channels). For example, intracellular calcium activates calmodulin, troponin and other proteins, which in turn activate enzymes that increase cyclic adenosine monophosphate (cAMP) and phosphorylation, triggering muscle contraction.

Ryanodine is a very large alkaloid of ˜5000 molecular weight. Ryanodine receptors (RyRs) are found in the sarcoplasmic reticulum (SR) in the interior of the cell and are closely associated physically and functionally with a voltage-gated calcium channels in cell membranes. The skeletal muscle ryanodine receptor (RyR1) is one of largest channels known. RyR is a tetramer, and a ligand-gated calcium channel. Each monomer has a mass of approximately 5,037 aa. (565 kda). In smooth muscle and cardiac muscle, the SR, which can contain an intracellular store of Ca²⁺, is located very close to the cell membrane (e.g. within approximately 200 angstrom of the cell membrane). This allows SR channels, such as ryanodine channels, to be easily activated by calcium flowing into the cell via voltage-sensitive calcium channels in the cell membrane, releasing an even larger amount of Ca²⁺ inside the cell via a process known as calcium-induced calcium release (CICR). The initial influx of calcium into the cell can be through cell membrane channels that are activated by an action potential that depolarizes the cell member, leading to the intracellular release of Ca²⁺ from the SR. This is an example of how a “global” change in calcium can set off a “local” change (also called a “calcium spark” or “calcium surge”) in calcium concentration inside the cell.

Cav1 calcium channels initiate excitation-contraction coupling in skeletal, cardiac and smooth muscles, including in the iris, and in the ciliary body in the eye. In skeletal muscles, however, intracellular calcium release occurs differently than as described above for smooth muscles and cardiac muscles. Entry of Ca²⁺ is required for excitation-contraction coupling in cardiac myocites, and Ca²⁺ entry via Cav1.2 triggers activation of the RyR2, and initiates CICR, activation of actomyosin and contraction. Although CICR was first discovered in skeletal muscles, Ca²⁺ entering the cell and activating the SR to release of more Ca²⁺ is not the primary mechanism of skeletal muscle calcium surges. One important mechanism of skeletal muscle calcium surges is transverse tubule (“t-tubule”) activation. In skeletal muscles, t-tubules are invaginations of the cell membrane that extend towards the inside of the cell, close to SR is located, as shown in FIG. 1C. An action potential depolarization travelling across the cell membrane that defines the t-tubule can activate the SR to release calcium inside the cell, without calcium entering the cell directly from the outside to activate the SR (although that process also occurs in skeletal muscles). Thus, for skeletal muscles, influx of Ca²⁺ is not required for calcium surge generation (and for resultant muscle contraction).

Thus, it would be useful to monitor Ca²⁺ concentration in both cytosolic media and the plasma in order to obtain meaningful kinetics of the muscle response to an excitation-contraction coupling stimulus. In smooth and cardiac muscle cells, opening of voltage gated calcium ion channels across the plasma membrane causes a surge of calcium ion current into the cellular media, covering a range of two orders of magnitude (×100) increase in Ca²⁺ concentration over a time scale of 10-100 milliseconds. One objective of this invention is to be able to detect and quantify such a surge in Ca²⁺ ion concentration in intracellular media even in the presence of other ions including Na⁺, K⁺ and Mg²⁺ that may interfere with the binding of Ca²⁺ ions on to a sensor surface, rendering the sensor less specific to the binding of Ca²⁺ ions. It is therefore useful to use coatings that are highly specific to Ca²⁺ for modification of the sensor surface. It is also desired that the sensor response be a simple (e.g., linear) function of free Ca²⁺ ions in the media around the sensor, so that simple signal processing may be used. The binding of Ca²⁺ ions on the modified sensor surface can be rapid, reaching equilibrium within 1 second, preferably within 100 milliseconds, and can be reversible, so that the equilibrium concentration of bound Ca²⁺ ions on the sensor surface is always dependent on the time dependent concentration of free Ca²⁺ ions in the intracellular media, thus providing an accurate measure of Ca²⁺ concentration.

Detection of Ca²⁺ ions by an implant, such as by a calcium sensor implant packaged with another implant (e.g. a knee implant, a hip implant, a brain implant, or a ocular implant), will serve a number of medical purposes. Ca²⁺ ions are linked to muscle contraction and relaxation, and concentration of Ca²⁺ ions or surges of Ca²⁺ ions can be indicative of the general health of an individual, or of nearby cells. For example, intracellular calcium release from the SR is required for cardiac muscle contraction. When the heart beats, the calcium concentration in the cytosol of cardiac muscle cells increase approximately ten fold from a resting molarity of approximately 10⁻⁷ to a molarity of approximately 10⁻⁶. A calcium deficiency (either a resting calcium deficiency or a deficient calcium surge strength) in cardiac muscles would impair the contractility of the muscle, and detection of such a deficiency would be beneficial in alerting an individual to a potential health problem. As another example, when implants such as knee implants or hip implants are surgically implanted in an individual, muscle near the surgery site is often accidentally or iatrogenically damaged. By monitoring calcium levels near the implant site, an individual's therapeutic recovery from the surgery can be monitored for abnormalities that may alert the individual or a medical professional to a potential failure of the implant to be integrated into tissue, thus causing health problems. A variety of medical conditions are correlated with abnormally high or abnormally low concentrations of calcium in a cell or in plasma near the cell. Thus, by detecting and/or monitoring calcium concentration levels in the cell or in the plasma near the cell, medical conditions can be detected and health professionals can be alerted to the problem.

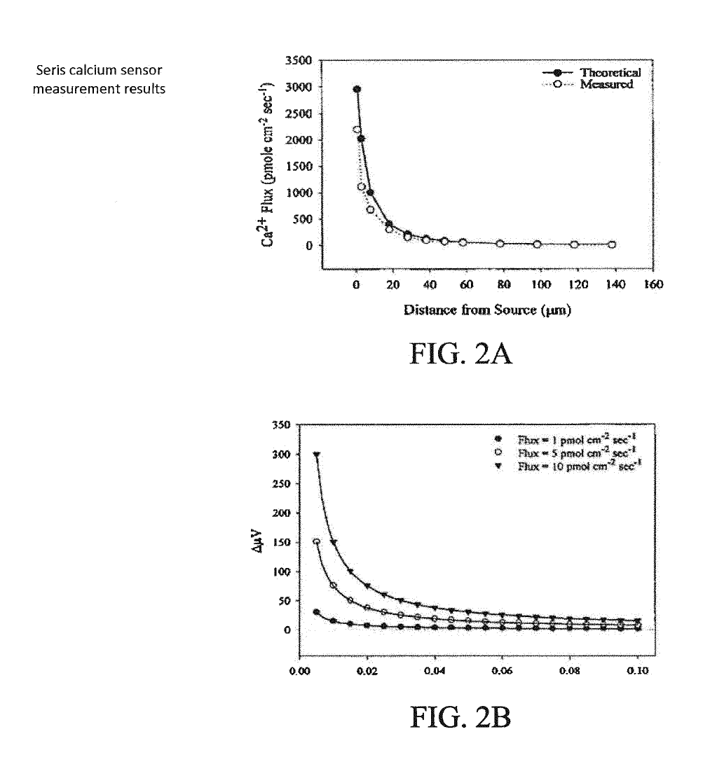

Detection and quantification of Ca²⁺ currents is well established in literature. For example, Smith, Hammer, Porterfield, and Sanger reported the development of micron size Ca²⁺ ion selective microelectrodes in order to measure Calcium ion flux across the interface between the cell and the plasma membrane. (Smith, P J S, Hammer, K, Porterfield, M, and sanger, R H, “Self-referencing, Non-invasive, Ion Selective Electrode for Single Cell Detection of Trans-Plasma Membrane Calcium Flux, in Microscopy Research and Technique, 46: 1999; pp 398-417). FIGS. 2A and 2B show results of calcium ion sensor measurements made using self-referencing ion selective (Seris) probes recorded by those authors. FIG. 2A shows the detection capability of the ion selective Ca2⁺ sensor, derivatized with a proprietary ligand, as reported by Smith, et al. FIG. 2B shows the detection capability of the ion selective microelectrode reported by Smith, et al.

While an ion selective microelectrode may have the capability to detect the calcium ion current in plasma cellular interfaces, such electrodes require careful assembly, use of an electrolyte that is best used fresh, and a detailed calibration procedure. Other methods to monitor in-situ calcium ion concentrations can represent improvements on this technique.

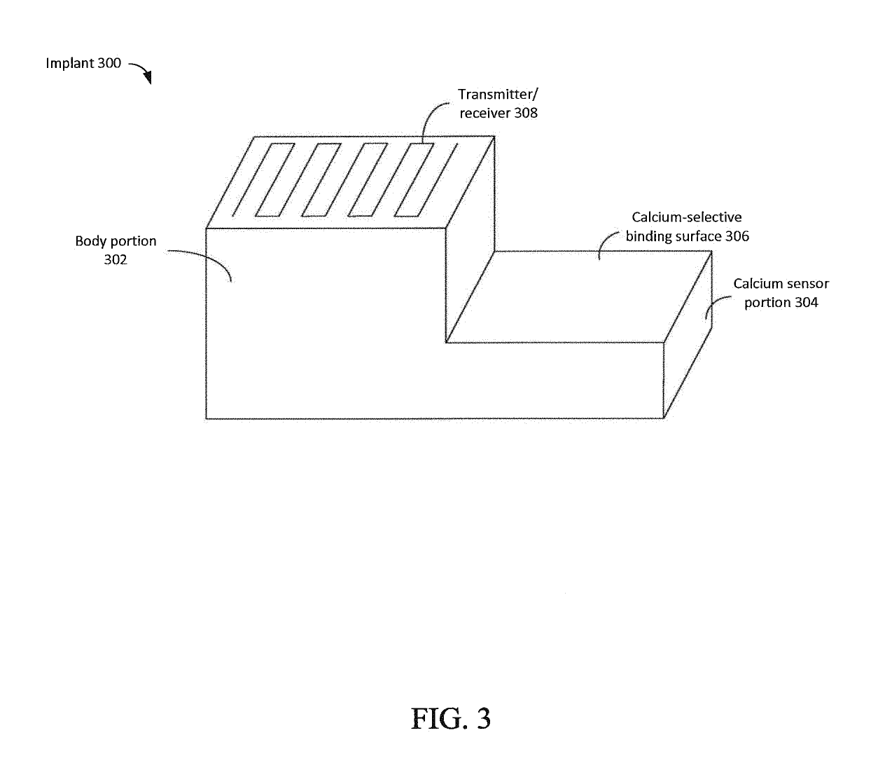

FIG. 3 depicts some embodiments of an implant 300. The implant 300 includes a body portion 302, a calcium sensor portion 304, and a transmitter/receiver 308. The calcium sensor portion 304 includes a calcium-selective binding surface 306.

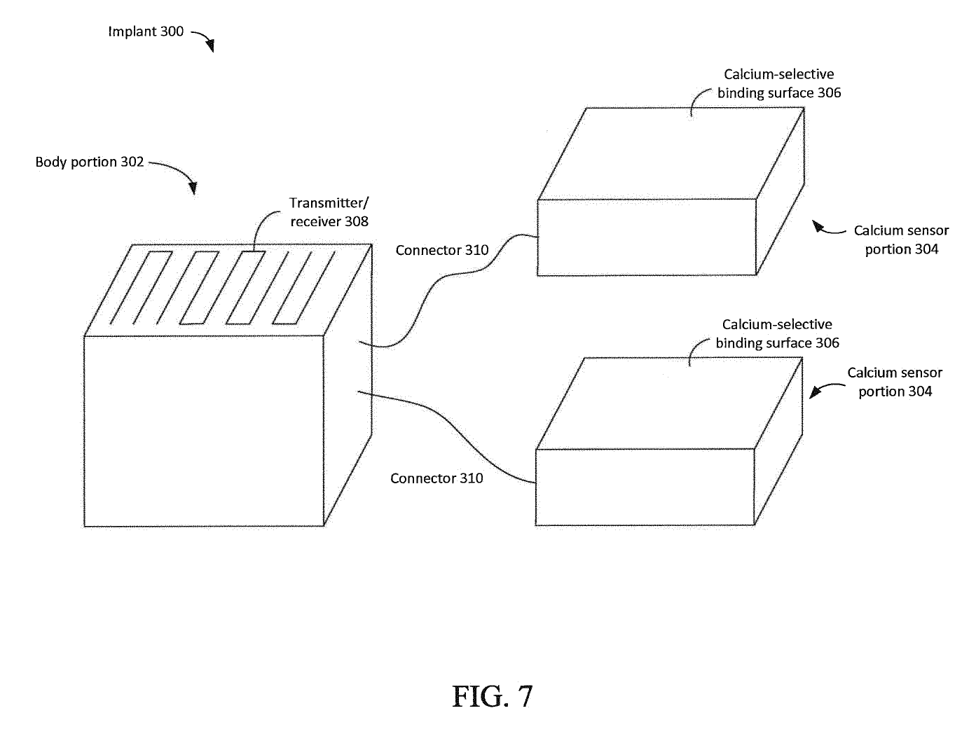

In the depicted embodiments, the implant 300 is in the shape of a parallelepiped having a step. That is, the implant 300 is in the shape of a parallelepiped wherein a portion of the parallelepiped is depressed such that the depressed portion is less extended in one direction (e.g. the vertical direction in the depicted implant 300) than is the non-depressed portion. In the depicted embodiments, the implant 300 includes the body portion 302, which is in the shape of a rectangular prism, and the calcium sensor portion 304, which is in the shape of a rectangular prism that is less extended in at least one direction than the body portion 302 (e.g. is shorter) and is disposed adjacent to, and is integrally connected with, the body portion 302. In other embodiments, such as the embodiments described below with respect to FIG. 7, the calcium sensor portion is not integrally connected to, or adjacent to, the body portion 302.

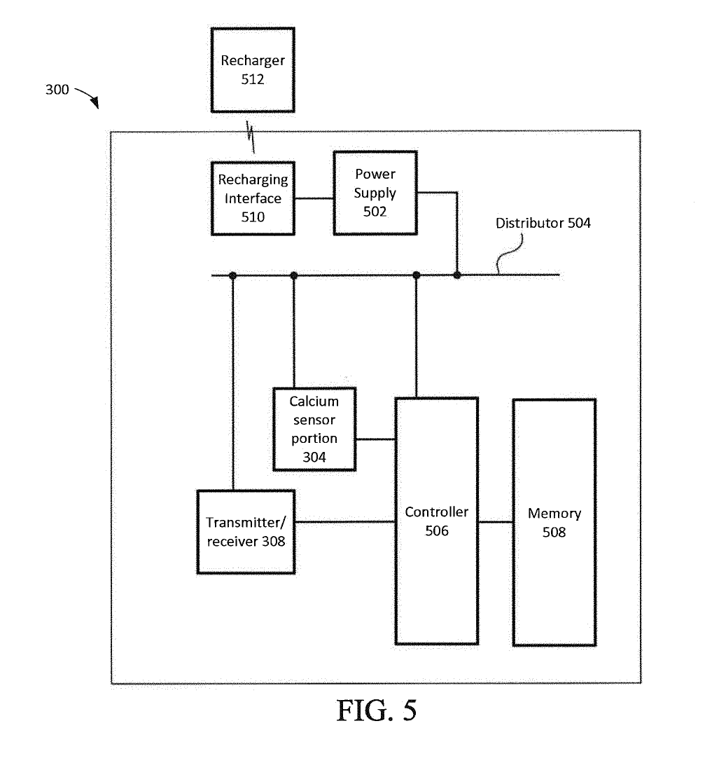

In some embodiments, the body portion 302 is an integrated circuit device that includes components such as a controller 506, a memory device 508, a distributor 504, a power supply 502, and a recharging interface 510, described in more detail below in reference to FIG. 5. The body portion 302 may include a substrate, which may include ceramic and/or metal, on which is disposed the above-mentioned components one or more plurality of layers. The components may be separated by insulation or passivation material, such as epoxy or resin layers, as appropriate to effectuate the electrical interactions between components described below in reference to FIG. 5. The body portion 302 may have dimensions of (5 millimeters±50%)×(1.5 millimeters±50%)×(1.5 millimeters±50%), and may preferably have dimensions of (5 millimeters±20%)×(1.5 millimeters±20%)×(1.5 millimeters±20%).

The body portion 302 may have a top surface (e.g. a surface that is not coplanar with any surface of the calcium sensor portion) on which is disposed a transmitter/receiver 308. The transmitter receiver 308 may be an antenna, and may include a trace, such as a metal trace. The trace may be, for example, a copper trace. The transmitter/receiver 308 may be configured to electronically communicate with the controller 506 via, for example, a printed circuit board (described in more detail below in reference to FIG. 5). The transmitter/receiver 308 may be configured to communicate with an external device, and may receive and/or transmit signals to an external device. For example, the transmitter/receiver 308 may communicate with an external wireless recharger device (described in more detail below in reference to FIG. 5) to power the implant 300, and/or may receive signals from an external device that cause the controller 506 to being data collection, and/or may transmit signals to an external device that include collected calcium ion data, such as calcium ion concentration data and/or calcium surge data.

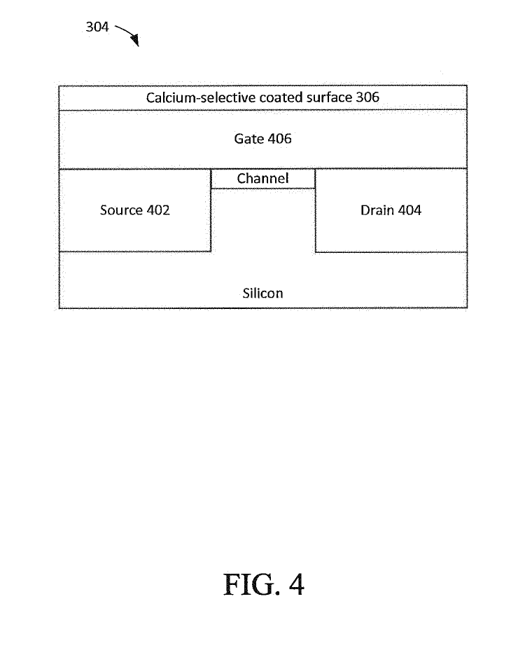

The calcium sensor portion 304 may be used to sense Ca²⁺ ions in its general vicinity. The calcium sensor portion 304 may have dimensions of (1 millimeter±50%)×(0.5 millimeters±50%)×(1.5 millimeters±50%), and may preferably have dimensions of (1 millimeters±20%)×(0.5 millimeters±20%)×(1.5 millimeters±20%). The calcium sensor portion 304 may include a calcium-selective binding surface 306 that can attract or bind Ca²⁺ ions. The calcium-selective binding surface 306 may be disposed on or located on a device that can transmit, generate, or alter a signal responsive to Ca²⁺ ions binding to the calcium-selective binding surface, such as, for example, a field effect transistor (FET), a cantilever, a microbalance, and an optical indicator portion of the calcium sensor portion 304 that includes a quantum dot laser configured to emit light based on changes in surface potential of the calcium-selective binding surface 306. Embodiments that include a FET are described below in reference to FIGS. 4, 9 and 10. Embodiments that include a cantilever are described in more detail below in reference to FIG. 11.

In some embodiments, the calcium-selective binding surface 306 may be disposed on a microbalance, such as a quartz microbalance. In a quartz microbalance, a standing wave can be established in a quartz crystal. If mass is added to the crystal, the resonant frequency of the standing wave changes. By disposing the calcium-selective binding surface 306 on the microbalance, Ca²⁺ ions bind to the surface and change the mass of the quartz crystal, resulting in a change in frequency of the standing wave. This change may be detected by circuitry in the calcium sensor portion, which can transmit a corresponding signal to the controller 506 indicative of the Ca²⁺ ion concentration bound to the calcium-selective binding surface 306.

In some embodiments, some embodiments, the calcium sensor portion 304 includes an optical indicator portion that includes a quantum dot laser configured to emit light based on changes in surface potential of a surface on which quantum dots are disposed. Thus, light may be generated based on Ca²⁺ ions binding to the calcium-selective binding surface 306. The optical indicator portion may further include a photodetector that can detect the light emitted by the quantum dot laser, and can responsively transmit a signal to the controller 506 indicative of calcium ion concentration.

The calcium-selective binding surface 306 may be a surface that attracts and/or binds Ca²⁺ ions. For example, the surface may include ionophores, amino acids, proteins, ligands, such as ethylene diamino tetra acetic acid (EDTA), or a ligand bound to a polymeric substrate, or a cryptand such as a cage molecule (e.g. a crown ether), that can transport or bind to Ca²⁺ ions. In other embodiments, the calcium-selective binding surface 306 may be coated with a calcium binding substance. For example, the calcium-selective binding surface 306 may be coated with a hydrogel or collagen material complexed with a substance having a high Ca²⁺ ion affinity. The binding coefficient of the substance should be significantly higher for Ca²⁺ than is the substance's binding coefficient for other ions found in plasma or inside a cell, such as potassium sodium or magnesium. Other ions may be present in the implant 300's environment in greater quantities than is found Ca²⁺, and for the calcium binding substance, the binding coefficient for Ca²⁺ should preferably be larger than the binding coefficient for potassium ions by a factor of at least 10⁶.

FIG. 4 depicts some embodiments of a calcium sensor portion 304. The depicted calcium sensor portion 304 includes a FET. The FET includes a source 402, a drain 404, a calcium-selective coated surface 306, and a gate 406. The calcium-selective coated surface 306 may be a collagen material complexed with a substance having a high Ca²⁺ ion affinity. As in any FET, the gate 406 controls a channel between the source 40 and the drain 404, and electrons flow across the channel. The electrical resistance at the channel is inversely proportional to its charge carrier density, which can be directly assess from a change in the source-drain voltage and current characteristics. Since the charge carrier density of the channel is sensitive to changes in the electric field in a direction perpendicular to the gate surface, the resistance can be correlated with both polarity and density of charges at the gate surface. When Ca²⁺ ions bind to the calcium-selective coated surface 306, the electrostatic potential of the gate changes, and the channel's resistance changes accordingly. This is reflected in, for example, drain to source voltage and current characteristics. The calcium sensor portion 304 can include circuitry to measure these characteristics and can output one or more signals to the controller 506 that correspond to those characteristics. Thus, Ca²⁺ ion concentration can be detected.

FIG. 5 is a block diagram depicting some embodiments of the implant 300. Specifically, FIG. 5 depicts electrical connectivity between some components of the implant 300. The depicted lines may be communication lines, power distribution lines, or any other manner of electric distribution. FIG. 5 is not meant to depict actual positions or relative positions of components of the implant 300, and those positions may vary. The depicted components include a power supply 502, a distributor 504, a controller 506, a memory device 508, a recharging interface 510, a recharger 512, the calcium sensor portion 304, and the transmitter/receiver 308. Other embodiments than those depicted may include fewer components, more components, and/or different components.

The power supply 502 can supply power to the calcium sensor portion 304, to the transmitter/receiver 308, and to the controller 506 via the distributor 504. The power supply includes one or more energy sources rechargeable through a recharging interface 510, described below. The power supply 502 may be, for example, a battery, such as a rechargeable lithium ion battery. The power supply 502 can include lithium-ceramic layers. The power supply 502 may be recharged, for example, through inductive charging using an external unit that provides energy to the implant 300. The power supply 502 may be a small battery, having dimensions of approximately 0.1 to approximately 20 mm×approximately 0.05 to approximately 18 mm×approximately 0.1 to approximately 10 mm, and preferably having dimensions of approximately 1.2 mm×approximately 0.8 mm×approximately 0.6 mm. In other embodiments, the battery may be larger or smaller than this. The battery can maintain a voltage at full charge in a range of approximately 3.0 volts (V) to approximately 4.1 V, and preferably maintains at full charge a voltage of approximately 4.0 to approximately 4.1 V·s, the voltages of the battery at full charge and/or full discharge may be larger or smaller than this.

The distributor 504 may electrically connect the two or more of the components of the implant 300. The distributor 504 may distribute, for example, power and/or communication signals. The distributor 504 may include more than one connection grid, such as, for example, an independent power distribution grid and an independent communications grid. In other embodiments, power and communications are distributed via the same grid. In some embodiments, a printed circuit board is used as a communications grid. In some embodiments, a solder bus is used for power distribution.

The controller 506 can be a logic controller, an application-specific integrated circuit (ASIC), a field-programmable gate array (FPGA), a microprocessor, a microcontroller, other circuitry effecting processor functionality, or a combination thereof, along with associated logic and interface circuitry. In some embodiments, the controller is a small ASIC, having dimension in a range of approximately 0.3 mm to approximately 3 mm×approximately 0.2 mm to approximately 2.6 mm×approximately 0.03 mm to approximately 1 mm, and preferably having dimensions of approximately 1.2 mm×approximately 0.8 mm×approximately 0.15 mm. In other embodiments, the controller may be larger or smaller than this.

The controller executes instructions, which may be implemented in hardware, firmware or software. For software-implemented instructions and some firmware-implemented instructions, the instructions can be stored in a memory device, which may be integrated into the controller, or may be external to the controller (such as memory device 508, described in more detail below). The memory device may be one or both of volatile and non-volatile memory for storing information (e.g., instructions and data). Examples of a memory device include a semiconductor memory device such as an EPROM, an EEPROM, a flash memory, a RAM, or a ROM device.

The controller 506 can receive power from the power supply 502 via, for example, the printed circuit board 504. The controller 506 can receive sensor data from the calcium sensor portion 304, and can store the sensor data in the memory device 508. The controller 506 can execute instructions stored in the memory device 508, and can transmit sensor data stored in the memory device 508 to an external device via the transmitter/receiver 308. The controller 506 can control power distribution in the implant 300, including controlling recharging processes described below.

The memory device 508 may store, for example, non-transitory computer-readable media having computer code thereon for performing various computer-implemented operations. The term “computer-readable storage medium” is used herein to include any medium that is capable of storing or encoding a sequence of instructions or computer codes for performing the operations, methodologies, and techniques described herein. Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel, and further transferred to the implant 300 either before or after implantation. Reprogrammability of the threshold actuating parameters coded into the firmware and stored in non-volatile memory inside the implant is an important feature of some embodiments that enable the surgeon or the caregiver to make it patient specific or customizable to a particular physiology.

The memory device 508 may include any combination of one or more EPROM, EEPROM, flash memory, RAM, or ROM devices. In some embodiments, the memory device 508 can include a flash device that stores firmware, and a RAM device that stores sensor data received from the calcium sensor portion 304.

The firmware may include code related to recharging procedures, data transmission procedures, or data processing procedures. For example, the firmware may include procedures related to a schedule for recharging the power supply 502 via the recharging interface 510. The firmware may include procedures related to a data transmission schedule that may be based on a time since last transmittal and/or an amount of yet-to-be-transmitted data stored in the memory device 508. The firmware may include data transmission procedures and/or recharging procedures that cause the controller to transmit data or commence a recharging procedure (discussed in more detail below) responsive to receiving a signal from an external source. The firmware may include a data processing procedure that, when executed by the controller 506, causes the controller 506 to determine whether a calcium surge was detected based on a signal output by the calcium sensor portion 304. In some embodiments, any of these procedures may be stored in the RAM device rather than in flash memory as firmware. For example, if it is expected that the data processing procedure may be updated, the data processing procedure may be stored on the RAM device.

The RAM device may store data, processed or unprocessed, transmitted by the calcium sensor portion 304. For example, the RAM device may store an indication that a surge has occurred. Such an indication may be timestamped, or otherwise associated with information indicating a time (absolute or relative to another time) at which the surge occurred or was noted. The RAM device may additionally or alternatively store a value related to a signal received from the calcium sensor portion 304, such as a voltage value or current value. Such values may be timestamped may be timestamped, or otherwise associated with information indicating a time (absolute or relative to another time) at which the signal having the value was received from the calcium sensor portion 304.

In some embodiments, the RAM device stores data collecting procedures. For example, the data collecting procedures may, when executed by the controller 506, cause the controller 506 to begin or stop storing data received from the calcium sensor portion 304. For example, the data collection procedures may cause the controller 506 to record data according to a data collection schedule, which may be part of the data collection procedures or may be stored in the firmware. The data collection procedures may cause the controller to not record data (e.g. may override collection of data according to the data collection schedule) when the controller determines that the power supply is low.

In some embodiments, the RAM device stores data processing procedures. For example, the data processing procedures may include time-averaging signal information, comparing values to predetermined (e.g. pre-programmed) thresholds, or thresholds determined based on “baseline” signals from secondary calcium sensor portions 304 (described in more detail below in reference to FIG. 7) to, for example, determine if an event such as a calcium surge has occurred, and/or to convert signal information received from the calcium sensor portion to a calcium concentration value, based on, for example, a predetermined correspondence between signal information and calcium concentration. In some embodiments, data processing procedures can involve comparing a detected calcium concentration to a reference or baseline calcium concentration. The reference or baseline calcium concentration may be, for example, a baseline calcium concentration determined based on a historical average concentration for an individual in which the implant 300 is implanted. The reference or baseline calcium concentration may be, for example, a baseline calcium concentration determined based on calcium concentration data corresponding to a second implant 300 implanted in an individual. For example, a first implant 300 and a second implant 300 may be implanted in different parts of an individual's body, and a calcium concentration detected by the second implant 300 may serve as a baseline calcium concentration for determining whether a calcium concentration detected by the first implant 300 falls within a “normal” or “healthy” range, e.g. is not too greatly deviated from the baseline concentration. In other embodiments, the baseline calcium concentration can be determined based on statistics of calcium concentration measurements taken from a plurality of individuals.

In some embodiments, the implant 300 includes the recharging interface 510. The recharging interface may include circuitry that includes an inductor or coil. The recharger 512 may be a device external to the implant 300 capable of directing or generating an electromagnetic field that generates current in the coil of the recharging interface 510. The recharging interface 510 may so draw power from the recharger 512 and supply that power to the power supply 502. The recharging interface 510 may supply power to the power supply 502 automatically when in the vicinity of the activated recharger 512, or may be primed to draw power via a signal from the controller 506, which may be output from the controller 506 responsive to the controller 506 receiving, via the transmitter/receiver 308, an activation signal from an external source. The recharger 512 may be operated to supply power to the recharging interface 510 by, for example, a healthcare professional, or by an individual in whom the implant 300 is implanted. This recharging process and circuitry can allow the implant 300 to have an extended shelf-life, without the need to surgically remove the implant 300 for charging or replacement.

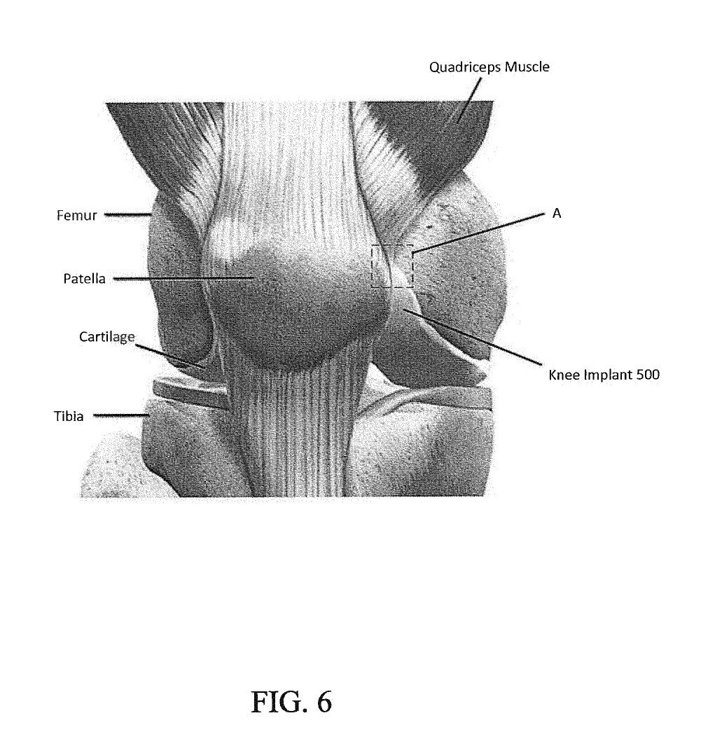

FIG. 6 depicts a knee implant 500 implanted in a person. A knee implant 500, as depicted, can be used, for example, to replace portions of a joint damaged by arthritis. The knee implant 500 can help to stop bones, such as the tibia and the femur, from rubbing together. As shown in FIG. 6, the knee implant 500 can be in contact with the quadriceps muscle. Portion A of the knee implant 500 shows a location where a full implant 300, packaged with the knee implant 500, can be located, or where a calcium sensor portion 304 of an implant 300 can be located. The calcium sensor portion 304 can function as a probe that detects the presence and/or concentration of Ca²⁺ ions. The calcium sensor portion 304 can be embedded in muscle tissue, such as muscle tissue near the knee implant 500, to detect intracellular and extracellular Ca²⁺ ions near or in local muscle cells. The calcium sensor portion 304 can be embedded in muscle tissue such that, over time, muscle tissue (such as muscle tissue damaged during surgery performed to implant the knee implant 500) grows over the implant calcium sensor portion 304, thus immersing the calcium sensor portion in plasma or cytosolic material, allowing the calcium sensor portion 304 to detect local Ca²⁺. In some embodiments, the implant 300 is connected to or made integral with another implant, such as the knee implant 500, or the implants described herein, or any other implant.

FIG. 7 depicts some embodiments of an implant 300 that includes more than one calcium sensor portion 304. In the depicted embodiments, two calcium sensor portions 304 are shown, but in other embodiments more than two calcium sensor portions 304 may be included in an implant 300. The calcium sensor portions 304 are each respectively electrically connected to the body portion 302 of the implant 300 by, for example, a flexible connector 310. The flexible connector 310 can be, for example, a micro flexible wire or cable.

In the depicted embodiments, the body portion 302 can act as a hub for two or more calcium sensor portions, and the controller 506 of the body portion 302 can process signals received from any connected calcium sensor portions 304. The calcium sensor portions 304 may be used as probes in this manner, and may be disposed in places of interest (e.g. may be embedded in muscle cells, such as muscle cells near a surgical implant site). By utilizing two or more calcium sensor portions 304, a differential calcium concentration or a calcium gradient can be measured. This can be used, for example, to compare calcium concentrations in different muscle cells. For example, one calcium sensor portions 304 may be embedded in a healthy cell and can generate a baseline “healthy” calcium concentration signal, while another calcium sensor portions 304 may be embedded in a cell proximate to a surgical site or in a cell that is otherwise desirable to monitor, and can detect any deviations from the measured healthy baseline signal. Multiple calcium sensor portions 304 can also be used to detect a calcium flow in plasma (e.g. calcium influx into a cell). Furthermore, by processing signals from multiple calcium sensor portions 304, noise or sensor malfunctions can be somewhat accounted for by, for example, having the controller 506 use an average detected calcium concentration level of the multiple calcium sensor portions 304.

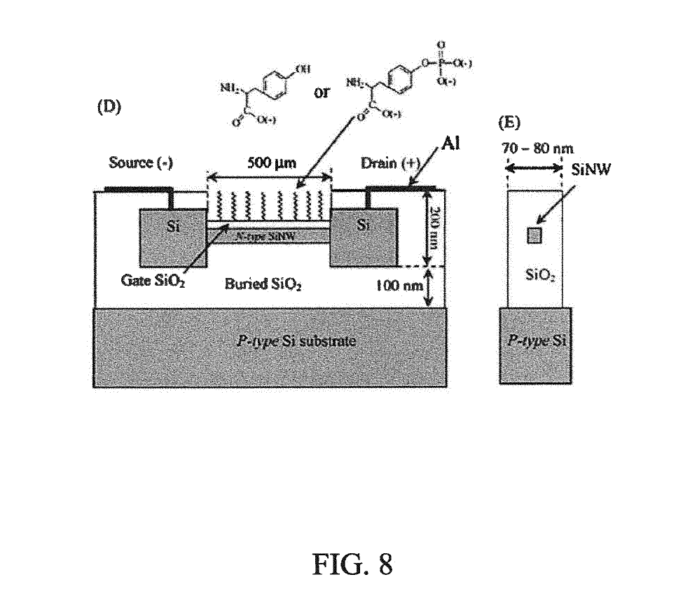

FIG. 8 shows a cross section of silicon nanowire based FET sensors for Ca2⁺ ion detection (Bi, X, Wong, W L, Ji, W, Agarwal, A, Balasubramanian, N, and Yang, K-L, “Development of electrochemical calcium ion sensors by using silicon nanowires modified with phosphotyrosine”, Biosensors and Bioelectronics, 23, 2008; pp 1442). The chemically modified silicon nanowires showed a change in their conductance as a function of Ca2⁺ concentration up to 10 μM, showing that cytosolic Ca2⁺ can change the positive gate voltage of an FET device made of n type silicon nanowires modified by coating with phosphotyrosine. Such a chemically modified nanowire can be used in a calcium sensor portion 304 of an implant 300 as a mechanism for detecting Ca2⁺ ions, similar to the manner described above with respect to FIG. 4.

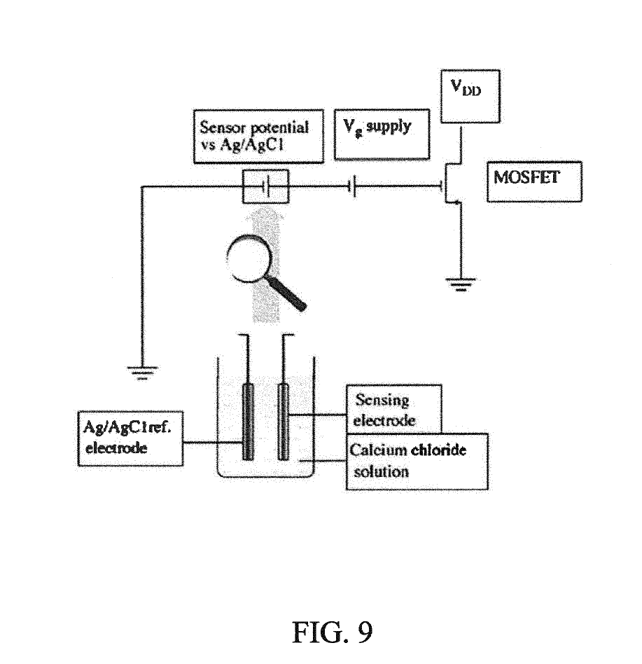

FIG. 9 shows an extended gate field effect transistor based on functionalized zinc oxide nanorods (Asif, M L, Nur, O, Willander, M, and Danielsson, B, “Selective calcium ion detection with functionalized ZnO nanorods-extended gate MOSFET”, in Biosensors and Bioelectronics, 24, 2009; pp 3379). In an extended gate field effect transistor, the current flowing from the source to the drain depends on the gate potential. The potential generated at the surface of the reference electrode can be added to the gate voltage, thus changing the source to drain current flow. Such an extended gate field effect transistor can be used in a calcium sensor portion 304, and the source to drain current can be output to the controller 506 as a calcium ion detection signal. The nanowires may include, for example, materials such as zinc oxides, silicon or graphene.

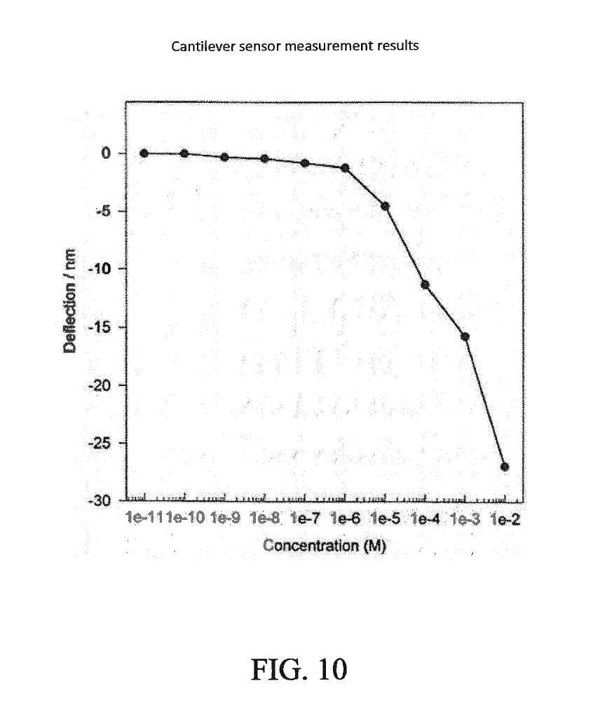

FIG. 10 shows cantilever sensor measurements recorded using a technique of micromechanical detection of trace amounts of calcium ions by using microcantilevers modified with ion selective self-assembled monolayers (SAMs). (Ji, H-F, Thundat, T, “In-situ detection of calcium ions with chemically modified microcantilevers” in Biosensors and Bioelectronics, 17, 2002; pp 337). The SAM modified microcantilevers undergo bending due to selective adsorption of calcium ions and are able to detect calcium ion concentrations in the range of nanomoles per liter. Such a microcantilever can constitute a calcium-selective coated surface 306 of a calcium sensor portion 304, and can be used in an implant 300 as a mechanism for detecting Ca2⁺ ions.

As used herein, the terms “approximately,” “substantially,” and “about” are used to account for small variations in dimensions or quantities. For example, when used in conjunction with a numerical value, the terms can encompass a range of variation of ±10% of that numerical value. As another example, an object can be referred to as being substantially in a shape if one or more dimensions of the object fall within ±10% of an extension that satisfies the definition of the shape (e.g. an object having dimensions of 1 mm×1 mm×1.05 mm can be said to be substantially in the shape of a cube). Definitions of shapes impose relative requirements on dimensions, and an object can be referred to as being substantially in a shape if, by first altering a first extension of the object in a first dimension ±10%, and then altering a second extension of the object in a second dimension by ±10%, the dimensions of the object can satisfy the definition of the shape.

What is claimed is:

1. An implant comprising:
a first calcium sensor portion comprising a calcium-selective binding portion; and
a body portion comprising:
a controller configured to process a signal received from the calcium-selective binding portion; and
a transmitter/receiver.

2. The implant of claim 1, wherein the first calcium sensor portion further comprises a field effect transistor, and wherein the calcium-selective binding portion is located on or electrically connected to a gate terminal or a channel of the field effect transistor.

3. The implant of claim 2, wherein the field effect transistor includes nanowire channels, and wherein the calcium-selective binding site is located on the nanowire channels.

4. The implant of claim 3, wherein the nanowire channels are composed of one or more materials including at least one of: silicon, a zinc oxide, or graphene.

5. The implant of claim 1, wherein the body portion has dimensions of (5 millimeters±50%)×(1.5 millimeters±50%)×(1.5 millimeters±50%), and the first calcium sensor portion has dimensions of (1 millimeter±50%)×(0.5 millimeters±50%)×(1.5 millimeters±50%).

6. The implant of claim 5, wherein the body portion has dimensions of (5 millimeters±20%)×(1.5 millimeters±20%)×(1.5 millimeters±20%), and the first calcium sensor portion has dimensions of (1 millimeter±20%)×(0.5 millimeters±20%)×(1.5 millimeters±20%).

7. The implant of claim 1, further comprising a second calcium sensor portion, wherein the first calcium sensor portion and the second calcium sensor portion are each respectively electronically connected to the body portion via a flexible electronic connector.

8. The implant of claim 1, further comprising a memory element, wherein the controller is further configured to store data corresponding to the processed signal in the memory device and to cause the transmitter/receiver to transmit the stored data.

9. The implant of claim 8, wherein the controller is configured to process a signal received from the first calcium sensor portion and to responsively store an indicia of whether a signal value of the signal is above a predetermined threshold.

10. The implant of claim 8, wherein the controller is configured to process a signal received from the first calcium sensor portion and to responsively store a voltage or current value of the signal.

11. The implant of claim 1, wherein the first calcium sensor portion further comprises a cantilever, and wherein the calcium-selective binding portion is located on a piezoelectric element of the cantilever.

12. The implant of claim 1, wherein the first calcium sensor portion further comprises an optical indicator portion, wherein the calcium selective-binding portion is located on the optical indicator portion, the optical indicator portion comprising a quantum dot laser configured to emit light based on changes in surface potential of the calcium-selective binding portion.

13. The implant of claim 1, wherein the first calcium sensor portion further comprises a microbalance, and wherein the calcium-selective binding portion is located on the microbalance.

14. The implant of claim 1, wherein the first calcium sensor portion further comprises an acoustic wave sensor, and wherein the calcium-selective binding portion is located on a surface of the acoustic wave sensor.

15. The implant of claim 1, wherein the controller is configured to transmit data corresponding to the processed signal to a caregiver or medical professional or database of a medial institution.

16. The implant of claim 1, further comprising a wirelessly rechargeable power source.

17. The implant of claim 1, wherein the wirelessly rechargeable power source is a lithium ion battery.

18. A calcium ion sensor comprising:
a calcium-selective binding portion;
a power source;
a transmitter/receiver;
a controller; and
a memory device.

19. The calcium ion sensor of claim 18, wherein:
the calcium ion sensor includes a field effect transistor,
the calcium-selective binding portion is located on or electrically connected to a gate terminal or a channel of the field effect transistor,
the transmitter/receiver includes a trace comprising at least one metal, and
the controller is configured to process a voltage signal received from the calcium-selective binding portion.

20. The calcium ion sensor of claim 19, wherein the calcium ion sensor has a volume less than 20 millimeters cubed.
